(19) United States
(12) Patent Application Publication
(10) Pub. No.: US 2021/0387021 A1
(43) Pub. Date: Dec. 16, 2021

(54) MASK FIXING APPARATUS, MASK COMPONENT AND RADIOTHERAPY DEVICE

(71) Applicants: OUR UNITED CORPORATION, Xi'an City (CN); Shenzhen OUR New Medical Technologies Development Co., Ltd., Shenzhen (CN)

(72) Inventors: Hui LIU, Xi'an City (CN); Daliang LI, Xi'an City (CN); Hao YAN, Xi'an City (CN); Peng ZAN, Xi'an City (CN)

(21) Appl. No.: 17/139,432

(22) Filed: Dec. 31, 2020

(30) Foreign Application Priority Data
Jun. 16, 2020 (CN) 202021113284.2
Jun. 16, 2020 (CN) 202021113291.2

(51) Int. Cl. A61N 5/10

(52) U.S. Cl. CPC A61N 5/1049; A61N 2005/1097

(57) ABSTRACT

A mask fixing apparatus, a mask component and a radiotherapy device are provided. The mask fixing apparatus includes: a frame, being configured to fix a mask, the frame being U-shaped having an inner boundary line and an outer boundary line, an opening of the frame fitting a neck of a patient, the inner boundary line of the frame being determined based on a periphery of a head of the patient, and the outer boundary line of the frame being determined based on a treatment cavity or an anti-collision cover in the treatment cavity; and a back-head support, being configured to support a back-head of the patient, and being connected with the frame to jointly carry the head of the patient, the inner surface of the back-head support fitting the back-head of the patient, and the outer surface of the back-head support being a curved surface having a preset curvature.

### CROSS-REFERENCE TO RELATED APPLICATIONS

This application claims the priority to Chinese Application No. 202021113284.2, entitled “Mask Fixing Apparatus and Radiotherapy Device adapted to a Radiotherapy System” filed on Jun. 16, 2020, and Chinese Application No. 202021113291.2, entitled “Mask Component adapted to a Radiotherapy System” filed on Jun. 16, 2020, both of which are hereby incorporated by references in its entirety.

### TECHNICAL FIELD

Embodiments of the present disclosure relate to the technical field of medical devices, and specifically to a mask fixing apparatus, a mask component, and radiotherapy device adapted for a radiotherapy system.

### BACKGROUND

Radiotherapy is an important means of cancer treatment, and it is usually necessary to fix a patient to avoid affecting the treatment effect due to the patient's movement during the radiotherapy. Fixation of the head of the patient is often achieved by using a mask. The mask is fixed on a mask fixing apparatus, and then the mask fixing apparatus is fixed to the radiotherapy system.

### SUMMARY

In view of this, one of the technical problems solved by embodiments of the present disclosure is to provide a mask fixing apparatus and a mask component adapted to a radiotherapy system, to overcome all or part of the problems of the radiotherapy device in the prior art.

An embodiment of the present disclosure provides a mask fixing apparatus adapted for a radiotherapy system. The mask fixing apparatus includes: a frame, being configured to fix a single-sided mask or a double-sided mask using a fixing mechanism, the frame being a U-shaped frame having an inner boundary line and an outer boundary line, an opening of the U-shaped frame fitting a neck of a patient, the inner boundary line of the U-shaped frame being determined based on a periphery of a head of the patient, and the outer boundary line of the U-shaped frame being determined based on a treatment cavity of the radiotherapy system or an anti-collision protective cover in the treatment cavity; and a back-head support, being configured to support a back-head of the patient, and being connected with the frame to jointly carry the head of the patient, the back-head support including an inner surface and an outer surface, the inner surface of the back-head support fitting the back-head of the patient, and the outer surface of the back-head support being a curved surface having a preset curvature.

Alternatively, a through hole is provided on the frame and the through hole cooperates with the fixing mechanism to fix the single-sided mask or the double-sided mask to the frame.

Alternatively, the fixing mechanism is a positioning pin or a locking member.

Alternatively, the back-head support has a left hollow area and a right hollow area.

Alternatively, the left hollow area has a left recess sub-area, the left recess sub-area is recessed in a direction of a central axis of the back-head support to enlarge a hollow area of the left hollow area; and the right hollow area has a right recess sub-area, and the right recess sub-area is recessed in the direction of the central axis of the back-head support to enlarge a hollow area of the right hollow area.

Alternatively, the back-head support has a top hollow area corresponding to a top of head of the patient.

Alternatively, the outer surface of the back-head support includes a first support portion having a first curvature, a second support portion having a second curvature, and a third support portion having a third curvature that are connected in sequence.

Alternatively, one end of the first support portion is connected to an end of the U-shaped frame opposite to the opening of the U-shaped frame, the other end of the first support portion is connected to one end of the second support portion, the other end of the second support portion is connected to one end of the third support portion and the other end of the third support portion is respectively connected to two end points at the opening of the U-shaped frame.

Alternatively, the first support portion, the second support portion, and the third support portion are an integrated structure.

Alternatively, the curvature of the second support portion is greater than the curvatures of the first support portion and the third support portion.

Alternatively, the curvature of the first support portion is greater than the curvature of the third support portion.

Alternatively, the preset curvature is determined based on the treatment cavity of the radiotherapy system or the anti-collision protective cover in the treatment cavity.

Alternatively, the back-head support further includes a boss protruding from the outer surface of the back-head support to the inner surface of the back-head support.

Alternatively, the boss includes at least one first boss and/or at least one second boss, the first boss extends along the first support portion and the second support portion, and the second boss extends along the third support portion.

Alternatively, the back-head support further includes at least one third boss protruding from the outer surface of the back-head support to the inner surface of the back-head support, the third boss is perpendicular to the first boss and traverses the back-head support.

Alternatively, the mask fixing apparatus further includes a headrest, and a positioning groove that cooperates with the boss for positioning is provided on the headrest.

Alternatively, the back-head support further includes a positioning hole provided thereon.

Alternatively, the mask fixing apparatus further includes a headrest, on which a positioning pin that cooperates with the positioning hole for positioning is provided.

Alternatively, a radius of the treatment cavity of the radiotherapy system or the anti-collision protective cover in the treatment cavity gradually changes along a central axis of the treatment cavity.

In another aspect, the present disclosure also provides a radiotherapy device, including the mask fixing apparatus according to any one of the above.

In yet another aspect, the present disclosure also provides a mask component adapted for a radiotherapy system, the mask component including:

a mask, including a main body area and an edge area, the main body area being adapted for a part where a patient needs to be fixed, and the edge area being connected to the main body area; and

a pressure plate, being a U-shaped structure having an inner boundary line and an outer boundary line, and the pressure plate detachably fixedly connecting the edge area of the mask to a mask fixing apparatus;

the pressure plate includes a first fixing portion and a second fixing portion;

the first fixing portion being located at a closed end of the U-shaped structure, and the first fixing portion including at least one first fixing position; and

the second fixing portion being located on both sides of an opening end of the U-shaped structure, the second fixing portion including at least two second fixing positions, the two second fixing positions being respectively located on both sides of a transverse centerline of the U-shaped structure, and a transverse distance between the two second fixing positions is 180 mm-220 mm.

Alternatively, the pressure plate further includes a third fixing portion located between the first fixing portion and the second fixing portion, the third fixing portion includes at least two third fixing positions, the two third fixing positions are respectively located on the both sides of the transverse centerline of the U-shaped structure, and a transverse distance between the two third fixing positions is 160 mm-210 mm.

Alternatively, the first fixing portion includes one first fixing position, and the first fixing position is located on a center line of the U-shaped structure; or,

the first fixing portion includes two first fixing positions, the two first fixing positions are respectively located on the both sides of the transverse centerline of the U-shaped structure, and a transverse distance between the two first fixing positions is 80-90 mm; or,

the first fixing portion includes three first fixing positions, where one of the first fixing positions is located on the center line of the U-shaped structure, and the other two of the first fixing positions are respectively located on the both sides of the transverse centerline of the U-shaped structure, and a transverse distance between the two first fixing positions is 80-90 mm.

Alternatively, the mask component further includes at least one adjustment gasket, the adjustment gasket is located between the pressure plate and the mask fixing apparatus; or,

the mask includes an upper mask and a lower mask, the mask component includes two pressure plates, the upper mask and the lower mask are respectively connected to one of the pressure plates, the mask component further includes at least one adjustment gasket, and the adjustment gasket is located between the upper mask pressure plate and the lower mask pressure plate.

The embodiments of the present disclosure use a U-shaped frame as the frame, and fix a single-sided or double-sided mask using a fixing mechanism. The opening of the U-shaped frame fits the neck of the patient, thereby ensuring that the frame is a complete frame except for the neck fitting opening, and only one medical staff is needed to achieve the fixation of the patient. The inner boundary line of the U-shaped frame is determined based on the periphery of the head of the patient, and the outer boundary line is determined based on the treatment cavity of the radiotherapy system, thereby ensuring that even a patient having a large head may use the mask fixing apparatus to realize radiotherapy in the treatment cavity of the radiotherapy system. The back-head support connected to the frame provides bearing capacity for the patient to prevent overhang of the mask fixing apparatus from affecting the accuracy of treatment. The inner surface of the back-head support fits the back-head of the patient, and the outer surface of the back-head support is a curved surface having a preset curvature, so that while performing whole-brain radiotherapy in the treatment cavity of the radiotherapy system, the patient avoids collisions with the treatment cavity of the radiotherapy system or the anti-collision protective cover in the treatment cavity.

### BRIEF DESCRIPTION OF THE DRAWINGS

Hereinafter, some specific embodiments of the embodiments of the present disclosure will be described in detail in an exemplary but not restrictive method with reference to the accompanying drawings. The same reference numerals in the accompanying drawings indicate the same or similar components or parts. Those skilled in the art should understand that these accompanying drawings are not necessarily drawn by ratio. In the accompanying drawings:

FIG. 1 is a schematic structural diagram of a mask fixing apparatus of a radiotherapy system;

FIGS. 2-7 are schematic structural diagrams of the mask fixing apparatus according to an embodiment of the present disclosure;

FIG. 8 is a schematic structural diagram of a mask component adapted for a radiotherapy system according to another embodiment of the present disclosure;

FIG. 9 is a schematic structural diagram of the mask component adapted for a radiotherapy system according to another embodiment of the present disclosure; and

FIGS. 10a and 10b are schematic structural diagrams of the mask component adapted for to a radiotherapy system according to another embodiment of the present disclosure.

The numerals in the accompanying drawings respectively indicate:
frame 22;
fixing mechanism 23;
mask fixing apparatus 3;
frame 31;
back-head support 11, 32;
first inner boundary line 311;
second inner boundary line 312;
third inner boundary line 313;
first outer boundary line 314;
second outer boundary line 315;
third outer boundary line 316;
through hole 317;
left hollow area 321;
right hollow area 322;
left recess sub-area 3211;
right recess sub-area 3221;
top hollow area 323;
first support portion 324;
second support portion 325;
third support portion 326;
first boss 327;
second boss 328;
third boss 329;
headrest 33;
positioning groove 331;
mask 4;
main body area 41;
edge area 42;
upper mask 43;
lower mask 44;
pressure plate 5;
first fixing portion 51;
first fixing position 511;
second fixing portion 52;
second fixing position 521;
third fixing portion 53;
third fixing position 531;
and adjustment gasket 6.

### DETAILED DESCRIPTION

The terms used in the present disclosure are only for the purpose of describing specific embodiments and are not intended to limit the present disclosure. The singular forms of “a”, “the” and “this” used in the present disclosure and the appended claims are also intended to include plural forms, unless the context clearly indicates otherwise. It should also be understood that the term “and/or” used herein refers to and includes any or all possible combinations of one or more associated listed items.

It should be understood that the “first”, “second” and similar words used in the specification and claims of the present disclosure do not denote any order, quantity or importance, but are only used to distinguish different components. Similarly, similar words such as “one” or “a” do not denote a quantity limit, but denote that there is at least one. Unless otherwise indicated, similar words such as “front”, “rear”, “lower” and/or “upper” are only for convenience of description, and are not limited to a position or a spatial orientation. “Comprising” or “including” and other similar words mean that the elements or items before “comprising” or “including” cover the elements or items listed after “comprising” or “including” and their equivalents, and do not exclude other elements or items. Similar words such as “connected” or “interconnected” are not limited to physical or mechanical connections, and may include electrical connections, whether direct or indirect.

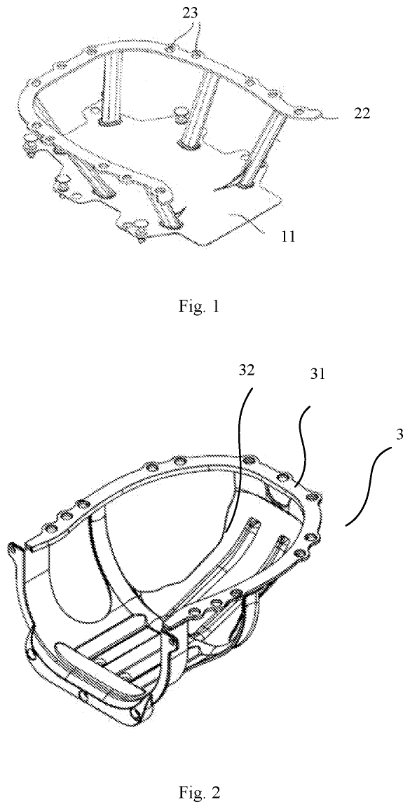

Referring to FIG. 1, a double-sided mask apparatus for fixing is used as an example, a spatial structure formed by the frame 22 of the mask fixing apparatus and the fixing mechanism 23 for fixing a mask occupies a large treatment space to accommodate the head of a patient, and an outer surface of the back-head support 11 supporting the back-head of the patient is flat. It is easy to collide with a treatment cavity of a radiotherapy system or an anti-collision protective cover in the treatment cavity during the radiotherapy.

The specific implementations of the embodiments of the present disclosure will be further described below in conjunction with the accompanying drawings of the embodiments of the present disclosure.

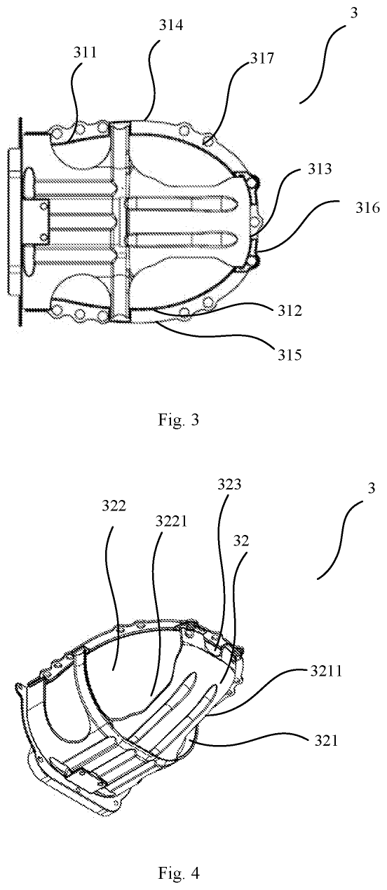

As shown in FIG. 2, an embodiment of the present disclosure provides the mask fixing apparatus 3 adapted for a radiotherapy system, and the mask fixing apparatus includes:

the frame 31, is configured to fix a single-sided mask or a double-sided mask using a fixing mechanism (not shown in the figure), the frame 31 is a U-shaped frame having an inner boundary line and an outer boundary line, an opening of the U-shaped frame fits the neck of a patient, the inner boundary line of the U-shaped frame is determined based on a periphery of the head of the patient, and the outer boundary line of the U-shaped frame is determined based on a treatment cavity of the radiotherapy system.

The back-head support 32, is configured to support the back-head of the patient, and is connected with the frame 31 to jointly carry the head of the patient, the back-head support 32 includes an inner surface and an outer surface, the inner surface of the back-head support 32 fits the back-head of the patient, and the outer surface of the back-head support 32 is a curved surface having a preset curvature.

The embodiment of the present disclosure uses a U-shaped frame as the frame, and fixes a single-sided or double-sided mask using a fixing mechanism. The opening of the U-shaped frame fits the neck of the patient, thereby ensuring that the frame is a complete frame except for the neck fitting opening, and only one medical staff is needed to achieve the fixation of the patient. The inner boundary line of the U-shaped frame is determined based on the periphery of the head of the patient, and the outer boundary line is determined based on the treatment cavity of the radiotherapy system, thereby ensuring that even a patient having a large head may use the mask fixing apparatus to realize radiotherapy in the treatment cavity of the radiotherapy system. The back-head support connected to the frame provides bearing capacity for the patient to prevent overhang of the mask fixing apparatus from affecting the accuracy of treatment. The inner surface of the back-head support fits the back-head of the patient, and the outer surface of the back-head support is a curved surface having a preset curvature, so that while performing whole-brain radiotherapy in the treatment cavity of the radiotherapy system, the patient avoids collisions with the treatment cavity of the radiotherapy system or the anti-collision protective cover in the treatment cavity.

In a specific implementation of the present disclosure, referring to FIG. 3, the inner boundary line of the U-shaped frame includes: the first inner boundary line 311, the second inner boundary line 312, and the third inner boundary line 313. The first inner boundary line 311 and the second inner boundary line 312 are respectively connected to the third inner boundary line 313, and the first inner boundary line 311 and the second inner boundary line 312 are symmetrically set at both ends of the third inner boundary line 313. The curvatures of the first inner boundary line 311 and the second inner boundary line 312 are the same, and the curvature of the first inner boundary line 311 and the curvature of the second inner boundary line 312 is smaller than the curvature of the third inner boundary line 313. The curvatures of the first inner boundary line 311, the second inner boundary line 312, and the third inner boundary line 313 are obtained by calculation according to different ages and different ethnic head models. Those skilled in the art may use any fitting calculation method to obtain, which is not limited in the present disclosure.

In another specific implementation of the present disclosure, referring to FIG. 3, the outer boundary line of the U-shaped frame includes: the first outer boundary line 314, the second outer boundary line 315, and the third outer boundary line 316. The first outer boundary line 314 and the second outer boundary line 315 are respectively connected to the third outer boundary line 316, and the first outer boundary line 314 and the second outer boundary line 315 are symmetrically set at both ends of the three outer boundary line 316. The curvatures of the first outer boundary line 314 and the second outer boundary line 315 are the same, and the curvature of the first outer boundary line 314 and the curvature of the second outer boundary line 315 is smaller than the curvature of the third outer boundary line 316. The curvatures of the first outer boundary line 314, the second outer boundary line 315, and the third outer boundary line 316 realize movement required for radiotherapy in the treatment cavity of the radiotherapy system based on the U-shaped frame, and are obtained by calculation based on fitting calculation that will not collide with the treatment cavity. Those skilled in the art may use any fitting calculation method to obtain, which is not limited in the present disclosure.

Referring to FIG. 3, a through hole 317 is provided on the frame 31, and the through hole 317 cooperates with the fixing mechanism (not shown in the figure) to fix the single-sided mask or the double-sided mask to the frame 31.

Specifically, the fixing mechanism (not shown in the figure) is a positioning pin or a locking member. The locking member may be a screw and a nut structure connected with the screw, an expansion locking mechanism (for example, an expansion plug and an expansion sleeve adapted for the expansion plug), a locking mechanism, and the like.

In the embodiment of the present disclosure, the single-sided mask or the double-sided mask is fixed to the frame through the provision of the through hole on the frame and the cooperation of the through hole with the positioning pin or the locking member. The operation is simple, and it is convenient to use, which can achieve good mask fixation effect.

In another specific implementation of the present disclosure, referring to FIG. 4, the back-head support 32 has the left hollow area 321 and the right hollow area 322.

In the embodiment of the present disclosure, the left hollow area and the right hollow area allow treatment rays to pass through the left hollow area and the right hollow area for brain treatment to avoid loss of therapeutic dose.

Specifically, the left hollow area 321 has the left recess sub-area 3211, and the left recess sub-area 3211 is recessed in a direction of a central axis of the back-head support 32 to enlarge a hollow area of the left hollow area 321; and the right hollow area 322 has the right recess sub-area 3221, and the right recess sub-area 3221 is recessed in the direction of the central axis of the back-head support 32 to enlarge a hollow area of the right hollow area 322.

In the embodiment of the present disclosure, the left recess sub-area and the right recess sub-area can increase the hollow areas of the left hollow area and the right hollow area without affecting the support to the back-head of the patient, thereby further reducing the possibility of collision between the back-head support and the treatment cavity of the radiotherapy system, which is beneficial for the mask fixing apparatus to realize focusing and other radiotherapy operations in the radiotherapy cavity of the radiotherapy system.

In another specific implementation of the present disclosure, referring to FIG. 4, the back-head support 32 has the top hollow area 323 corresponding to the top of head of the patient.

In the embodiment of the present disclosure, the top hollow area is provided on the back-head support at the top of head position of the patient, which can also reduce stress concentration during the processing of the mask fixing apparatus and ensure the quality of the mask fixing apparatus.

In another specific implementation of the present disclosure, referring to FIG. 5, the outer surface of the back-head support 32 includes the first support portion 324 having a first curvature, the second support portion 325 having a second curvature, and the third support portion 326 having a third curvature that are connected in sequence.

Alternatively, one end of the first support portion 324 is connected to the center point of an opposite end of the opening of the U-shaped frame, and the other end of the first support portion 324 is connected to one end of the second support portion 325. The other end of the second support portion 325 is connected to one end of the third support portion 326, and the other two ends of the third support portion 326 are respectively connected to two end points at the opening of the U-shaped frame.

Alternatively, the first support portion 324, the second support portion 325, and the third support portion 326 are an integrated structure.

Alternatively, the curvature of the second support portion 325 is greater than the curvature of the first support portion 324 and the curvature of the third support portion 326.

In the embodiment of the present disclosure, the second support portion corresponds to a position between the top of the head and the back neck of the patient, and is likely to collide with the radiotherapy cavity when performing radiotherapy operations such as focusing in the radiotherapy cavity of the radiotherapy system. In addition, the curvature of the position between the top of the head and the back neck of the patient is relatively large, so the curvature of the second support portion is greater than that of the first support portion and the third support portion, so as to further prevent the back-head support from colliding with the radiotherapy cavity during the radiotherapy.

Specifically, the curvature of the first support portion 324 is greater than the curvature of the third support portion 326.

Since the first support portion corresponds to the top of head position of the patient, and the third support portion is adapted for the back neck position of the patient's head, the third support portion has a smaller curvature than the first support portion, so as to ensure a better fit to the back neck of the patient.

Alternatively, the preset curvature is determined based on the treatment cavity of the radiotherapy system or the anti-collision protective cover in the treatment cavity.

Therefore, the embodiment of the present disclosure can further prevent the back-head support from colliding with the treatment cavity of the radiotherapy system or the anti-collision protective cover in the treatment cavity during the radiotherapy.

In another specific implementation of the present disclosure, referring to FIG. 6, the back-head support 32 further includes a boss protruding from the outer surface of the back-head support 32 to the inner surface of the back-head support 32.

The boss includes at least one first boss 327 and/or at least one second boss 328, the first boss 327 extends along the first support portion 324 and the second support portion 325, and the second boss 328 extends along the third support portion 326.

In another specific implementation of the present disclosure, referring to FIG. 6, the back-head support 32 further includes at least one third boss 329 protruding from the outer surface of the back-head support 32 to the inner surface of the back-head support 32, the third boss 329 is perpendicular to the first boss 327 and traverses the back-head support 32. Specifically, the traverse includes that both ends of the third boss 329 are connected to the frame 31 or both ends of the third boss 329 are not connected to the frame 31.

In the embodiment of the present disclosure, the bosses can be used as reinforcing ribs to further improve the bearing capacity of the back-head support; at the same time, when the mask fixing apparatus 3 includes a headrest, the first boss and/or the second boss and/or the third boss can also be used as a positioning part of the headrest for headrest positioning, that is: the first boss and/or the second boss is used to limit the headrest in the left-right direction, and the third boss is used to limit the headrest in the front-back direction.

In another specific implementation of the present disclosure, referring to FIG. 7, the mask fixing apparatus 3 further includes the headrest 33, and the headrest 33 is provided with the positioning groove 331 that cooperates with the first boss 327 and/or the second boss 328 and/or the third boss 329 for positioning.

Specifically, the back-head support 32 further includes a positioning hole provided thereon (not shown in the figure).

In another specific implementation of the present disclosure, the mask fixing apparatus 3 further includes the headrest 33, on which a positioning pin (not shown in the figure) that cooperates with the positioning hole for positioning is provided. The pin hole positioning method ensures the reliability of the headrest positioning.

In another specific implementation of the present disclosure, a radius of the treatment cavity of the radiotherapy system or the anti-collision protective cover in the treatment cavity gradually changes along a central axis of the treatment cavity. Here, it should be noted that the mask fixing apparatus provided in the embodiments of the present disclosure may be applicable to radiotherapy device with any shape or structure of the treatment cavity or the anti-collision protective cover, and is not limited to radiotherapy device whose radius gradually changes along the central axis of the treatment cavity.

Exemplarily, the treatment cavity of a gamma knife radiotherapy system is cone-shaped or hemispherical, because when the cone-shaped or hemispherical treatment cavity extends from the entrance of the treatment cavity to the inside of the treatment cavity in the direction of the centerline of the treatment cavity, the radius of the treatment cavity gradually reduces, so that the mask fixing apparatus is likely to collide with the treatment cavity of the radiotherapy system or a protective cover of the treatment cavity of the radiotherapy system. The outer surface of the back-head support of the present disclosure is a curved surface having a preset curvature, so that while performing whole-brain radiotherapy in the treatment cavity of the radiotherapy system, the patient avoids collisions with the treatment cavity of the radiotherapy system or the anti-collision protective cover in the treatment cavity.

An embodiment of the present disclosure also provides a radiotherapy device, including the mask fixing apparatus according to any one of the above.

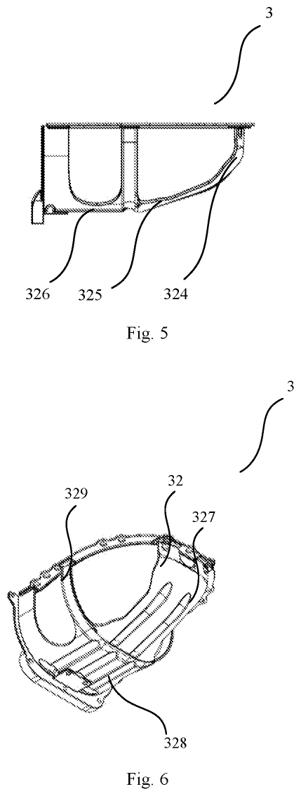

As shown in FIG. 8, an embodiment of the present disclosure provides a mask component adapted for a radiotherapy system, and the mask component includes: the mask 4, including the main body area 41 and the edge area 42, the main body area 41 is adapted for a part where a patient needs to be fixed, and the edge area 42 is connected to the main body area 41; and the pressure plate 5, is a U-shaped structure having an inner boundary line and an outer boundary line, and the pressure plate 5 detachably fixedly connects the edge area 42 of the mask 4 to the mask fixing apparatus; the pressure plate 5 includes the first fixing portion 51 and the second fixing portion 52; the first fixing portion 51 is located at a closed end of the U-shaped structure, the first fixing portion 51 includes at least one first fixing position 511; and the second fixing portion 52 is located on both sides of an opening end of the U-shaped structure, the second fixing portion 52 includes at least two second fixing positions 521, the two second fixing positions 521 are respectively located on both sides of a transverse centerline of the U-shaped structure, and a transverse distance between the two second fixing positions 521 is 180 mm-220 mm.

In the embodiment of the present disclosure, the mask component adapted for a radiotherapy system includes: the mask and the pressure plate. The pressure plate includes the first fixing portion and the second fixing portion. The mask includes the connected main body area and edge area. The first fixing portion is located at the closed end of the U-shaped structure. The first fixing portion includes at least one first fixing position. The second fixing portion is located on both sides of the opening end of the U-shaped structure, the second fixing portion includes at least two second fixing positions, the two second fixing positions are respectively located on both sides of the transverse centerline of the U-shaped structure, and the transverse distance between the two second fixing positions is 180 mm-220 m. Therefore, the embodiment of the present disclosure can achieve good fixation of the mask on the head of the patient through the fixation position provided on the pressure plate, and avoid affecting the effect of radiotherapy because the head of the patient cannot be well fixed.

Specifically, the first fixing position 511 and/or the second fixing positions 521 are positioning pin positions or locking member positions.

Using the positioning pin positions or the locking member positions as the first fixing position and/or the second fixing positions can facilitate assembly and improve a fastening effect.

In another specific implementation of the present disclosure, the pressure plate 5 further includes a third fixing portion 53 located between the first fixing portion 51 and the second fixing portion 52, the third fixing portion 53 includes at least two third fixing positions 531, the two third fixing positions 531 are respectively located on the both sides of the transverse centerline of the U-shaped structure, and a transverse distance between the two third fixing positions 531 is 160 mm-210 mm.

In the embodiment of the present disclosure, the third fixing portion is set between the first fixing portion and the second fixing portion, and the transverse distance of the third fixing portion between the two third fixing portion is 80-90 mm, so as to ensure a better fixing effect on the mask component.

Specifically, the third fixing positions 531 are positioning pin positions or locking member positions.

Using the positioning pin positions or the locking member positions as the third fixing positions can facilitate assembly and improve a fastening effect.

In another specific implementation of the present disclosure, the first fixing portion 51 includes one first fixing position 511.

In the embodiment of the present disclosure, only one first fixing position 511 is provided, which reduces tooling costs and simplifies the manufacturing process.

In another specific implementation of the present disclosure, the first fixing position 51 is located on a center line of the U-shaped structure.

In the embodiment of the present disclosure, as the first fixing position is located on the center line of the U-shaped structure, a more stable fixing effect on the mask component can be achieved.

In another specific implementation of the present disclosure, the first fixing portion 51 includes two first fixing positions 511, the two first fixing positions 511 are respectively located on the both sides of the transverse centerline of the U-shaped structure, and a transverse distance between the two first fixing positions 511 is 80-90 mm.

In the embodiment of the present disclosure, by setting two first fixing positions, and the two first fixing positions are respectively located on the both sides of the transverse centerline of the U-shaped structure, and the transverse distance between the two first fixing positions is 80-90 mm, a more stable fixing effect on the mask component can be achieved.

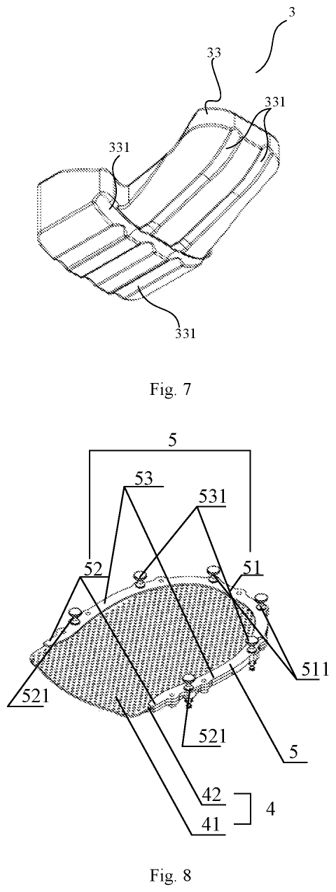

In another specific implementation of the present disclosure, referring to FIG. 9, the mask component further includes at least one adjustment gasket 6, the adjustment gasket is located between the pressure plate 5 and the mask fixing apparatus.

In the embodiment of the present disclosure, when the mask component is fixed to the mask fixing apparatus by the adjustment gasket between the pressure plate and the mask fixing apparatus, the tension of the mask is adjusted by the adjustment gasket, which facilitates the assembly operation.

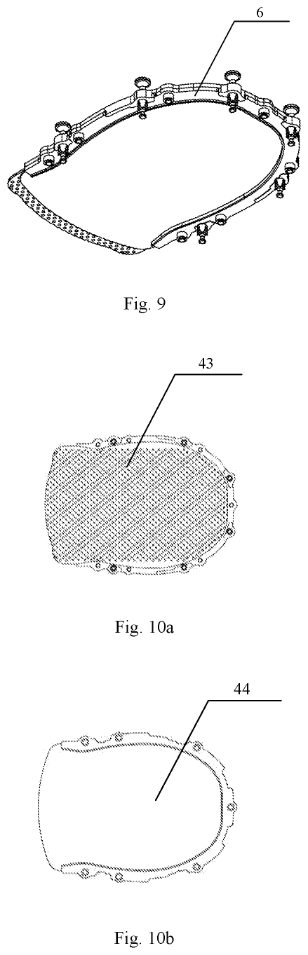

In another specific implementation of the present disclosure, referring to FIG. 10a and FIG. 10b, the mask 4 includes the upper mask 43 and the lower mask 44, the mask component includes two pressure plates, the upper mask 43 and the lower mask 44 are respectively connected to one pressure plates 5.

In the embodiment of the present disclosure, with the two pressure plates, the upper mask and the lower mask can be more stably fixed to the mask fixing apparatus, and a better mask fixing effect can be achieved.

In another specific implementation of the present disclosure, the mask component further includes at least one adjustment gasket, and the adjustment gasket is located between the upper mask pressure plate and the lower mask pressure plate.

In the embodiment of the present disclosure, when the mask component is fixed to the mask fixing apparatus by the adjustment gasket between the upper mask pressure plate and the lower mask pressure plate, the tension of the mask is adjusted by the adjustment gasket, which facilitates the assembly operation.

In another specific implementation of the present disclosure, the edge area 42 of the mask 4 is detachably fixedly connected to the pressure plate 5 by welding.

In another specific implementation of the present disclosure, by providing the through holes in the edge area 42 of the mask 4, the pressure plate 5 and the edge area 42 of the mask 4 are detachably fixedly connected.

Specifically, the detachably fixed connection includes directly welding the edge area 42 of the mask 4 on the pressure plate 5 or by piercing the edge area 42 of the mask 4, and the piercing corresponds to the first fixing position 511 and the second fixing positions 521. The pressure plate 5 realizes the fixed connection of the edge area 42 of the mask 4 through the first fixing position 511, the second fixing positions 521, and piercing to match the apparatus for fixing.

The embodiments in this specification are described in a progressive way, and the same or similar parts between the various embodiments can be referred to each other, and each embodiment focuses on the differences from other embodiments. In particular, as for the system embodiment, since it is basically similar to the method embodiment, the description is relatively simple, and for related parts, reference may be made to the parts of the description of the method embodiment.

The above descriptions are only examples of the present disclosure and are not used to limit the present disclosure. For those skilled in the art, the present disclosure may have various modifications and changes. Any modification, equivalent replacement, improvement, etc. made within the spirit and principle of the present disclosure shall be included in the scope of the claims of the present disclosure.

What is claimed is:

1. A mask fixing apparatus adapted for a radiotherapy system, the mask fixing apparatus comprising:
a frame, being configured to fix a single-sided mask or a double-sided mask using a fixing mechanism, the frame being a U-shaped frame having an inner boundary line and an outer boundary line, an opening of the U-shaped frame fitting a neck of a patient, the inner boundary line of the U-shaped frame being determined based on a periphery of a head of the patient, and the outer boundary line of the U-shaped frame being determined based on a treatment cavity of the radiotherapy system or an anti-collision protective cover in the treatment cavity; and
a back-head support, being configured to support a back-head of the patient, and being connected with the frame to jointly carry the head of the patient, the back-head support comprising an inner surface and an outer surface, the inner surface of the back-head support fitting the back-head of the patient, and the outer surface of the back-head support being a curved surface having a preset curvature.

2. The mask fixing apparatus according to claim 1, wherein a through hole is provided on the frame, and the through hole cooperates with the fixing mechanism to fix the single-sided mask or the double-sided mask to the frame.

3. The mask fixing apparatus according to claim 1, wherein the back-head support has a left hollow area and a right hollow area.

4. The mask fixing apparatus according to claim 3, wherein the left hollow area has a left recess sub-area, the left recess sub-area is recessed in a direction of a central axis of the back-head support to enlarge a hollow area of the left hollow area; and
the right hollow area has a right recess sub-area, and the right recess sub-area is recessed in the direction of the central axis of the back-head support to enlarge a hollow area of the right hollow area.

5. The mask fixing apparatus according to claim 4, wherein the back-head support has a top hollow area corresponding to a top of head of the patient.

6. The mask fixing apparatus according to claim 1, wherein the outer surface of the back-head support comprises a first support portion having a first curvature, a second support portion having a second curvature, and a third support portion having a third curvature that are connected in sequence.

7. The mask fixing apparatus according to claim 6, wherein one end of the first support portion is connected to an end of the U-shaped frame opposite to the opening of the U-shaped frame, an other end of the first support portion is connected to one end of the second support portion, an other end of the second support portion is connected to one end of the third support portion and an other end of the third support portion is respectively connected to two end points at the opening of the U-shaped frame.

8. The mask fixing apparatus according to claim 7, wherein the curvature of the second support portion is greater than the curvatures of the first support portion and the third support portion, and the curvature of the first support portion is greater than the curvature of the third support portion.

9. The mask fixing apparatus according to claim 1, wherein the preset curvature is determined based on the treatment cavity of the radiotherapy system or the anti-collision protective cover in the treatment cavity.

10. The mask fixing apparatus according to claim 6, wherein the back-head support further comprises a boss protruding from the outer surface of the back-head support to the inner surface of the back-head support.

11. The mask fixing apparatus according to claim 10, wherein the boss comprises at least one of a first boss and a second boss, the first boss extends along the first support portion and the second support portion, and the second boss extends along the third support portion.

12. The mask fixing apparatus according to claim 11, wherein the back-head support further comprises at least one third boss protruding from the outer surface of the back-head support to the inner surface of the back-head support, the third boss is perpendicular to the first boss and traverses the back-head support.

13. The mask fixing apparatus according to claim 12, wherein the mask fixing apparatus further comprises a headrest, and a positioning groove that cooperates with the boss for positioning is provided on the headrest.

14. The mask fixing apparatus according to claim 1, wherein the back-head support further comprises a positioning hole provided thereon, the mask fixing apparatus further comprises a headrest, on which a positioning pin that cooperates with the positioning hole for positioning is provided.

15. The mask fixing apparatus according to claim 1, wherein a radius of the treatment cavity of the radiotherapy system or the anti-collision protective cover in the treatment cavity gradually changes along a central axis of the treatment cavity.

16. A radiotherapy device, comprising a mask fixing apparatus, the mask fixing apparatus further comprising:
a frame, being configured to fix a single-sided mask or a double-sided mask using a fixing mechanism, the frame being a U-shaped frame having an inner boundary line and an outer boundary line, an opening of the U-shaped frame fitting a neck of a patient, the inner boundary line of the U-shaped frame being determined based on a periphery of a head of the patient, and the outer boundary line of the U-shaped frame being determined based on a treatment cavity of the radiotherapy system or an anti-collision protective cover in the treatment cavity; and
a back-head support, being configured to support a back-head of the patient, and being connected with the frame to jointly carry the head of the patient, the back-head support comprising an inner surface and an outer surface, the inner surface of the back-head support fitting the back-head of the patient, and the outer surface of the back-head support being a curved surface having a preset curvature.

17. A mask component adapted for a radiotherapy system, the mask component comprising:
a mask, comprising a main body area and an edge area, the main body area being adapted for a part where a patient needs to be fixed, and the edge area being connected to the main body area; and
a pressure plate, being a U-shaped structure having an inner boundary line and an outer boundary line, and the pressure plate detachably fixedly connecting the edge area of the mask to a mask fixing apparatus;
the pressure plate further comprising:
a first fixing portion, the first fixing portion being located at a closed end of the U-shaped structure, and the first fixing portion comprising at least one first fixing position; and
a second fixing portion, the second fixing portion being located on both sides of an opening end of the U-shaped structure, the second fixing portion comprising at least two second fixing positions, the two second fixing positions being respectively located on both sides of a transverse centerline of the U-shaped structure, and a transverse distance between the two second fixing positions is 180 mm-220 mm.

18. The mask component according to claim 17, wherein the pressure plate further comprises a third fixing portion located between the first fixing portion and the second fixing portion, the third fixing portion comprises at least two third fixing positions, the two third fixing positions are respectively located on the both sides of the transverse centerline of the U-shaped structure, and a transverse distance between the two third fixing positions is 160 mm-210 mm.

19. The mask component according to claim 18, wherein the first fixing portion comprises one first fixing position, and the first fixing position is located on a center line of the U-shaped structure; or,
the first fixing portion comprises two first fixing positions, the two first fixing positions are respectively located on the both sides of the transverse centerline of the U-shaped structure, and a transverse distance between the two first fixing positions is 80-90 mm; or,
the first fixing portion comprises three first fixing positions, wherein one of the first fixing positions is located on the center line of the U-shaped structure, and the other two of the first fixing positions are respectively located on the both sides of the transverse centerline of the U-shaped structure, and a transverse distance between the two first fixing positions is 80-90 mm.

20. The mask component according to claim 17, wherein the mask component further comprises at least one adjustment gasket, the adjustment gasket is located between the pressure plate and the mask fixing apparatus; or,
the mask comprises an upper mask and a lower mask, the mask component comprises two said pressure plates, the upper mask and the lower mask are respectively connected to one of the pressure plates, the mask component further comprises at least one adjustment gasket, and the adjustment gasket is located between the upper mask pressure plate and the lower mask pressure plate.
